(19) United States
(12) Patent Application Publication
(10) Pub. No.: US 2009/0202034 A1
(43) Pub. Date: Aug. 13, 2009

(54) Method for three-dimensional image reconstruction of a dynamically moved object from data of an imaging device as well as associated imaging device

(75) Inventors: Gunter Lauritsch, Erlangen (DE); Alois Nottling, Pottenstein (DE); Christopher Rohkohl, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 12/321,909

(22) Filed: Jan. 27, 2009

(30) Foreign Application Priority Data
Feb. 12, 2008 (DE) 10 2008 008 611.8

(51) Int. Cl. H05G 1/60; A61B 5/05

(52) U.S. Cl. 378/4; 600/425

(57) ABSTRACT

The invention relates to a method for three-dimensional image reconstruction of a dynamically moved object from projection data of an imaging device comprising: a) determining a reference volume from the projection data which simulates a similar static object instead of the dynamically moved object; b) assigning the projection data to at least two disjoint consistent subsets, with a volume part reconstruction of the dynamically moved object being undertaken for each subset from projection data assigned to the subsets; c) applying a transformation to the respective volume part reconstructions representing a dynamic movement of the object; d) comparing the volumes of the transformed part reconstructions with a corresponding part of the reference volume, whereby, depending on the comparison, result step c) may be at least partly repeated; and e) summing the volume part reconstructions resulting from step c) and d) to form an overall three-dimensional image reconstruction.

### CROSS REFERENCE TO RELATED APPLICATIONS

This application claims priority of German application No. 10 2008 008 611.8 filed Feb. 12, 2008, which is incorporated by reference herein in its entirety.

### FIELD OF THE INVENTION

The invention relates to a method for three-dimensional image reconstruction of a dynamically moved object from projection data of an imaging device as well as to an associated imaging device, especially a conventional x-ray device, a C-arm x-ray device or a computer tomography device.

### BACKGROUND OF THE INVENTION

For image reconstruction of x-ray projection data, primarily in computer tomography, the primary method employed is Filtered Back Projection (FBP). FBP is based on the assumption that there is a stationary object in the projection data. In filtered back projection for static objects each individual projection of the projection data is filtered after corresponding preprocessing with a convolution core and projected back into the object space. The sum of these back projections produces the reconstruction of the sought object. For non-stationary objects this assumption leads to movement artifacts such as duplicated objects, stripes or to a low contrast for example.

Especially in the reconstruction of an if necessary contrasted part of a coronary vessel system—the coronary sinus and the adjacent branch-off vessels, there are two main sources of movement in the form of the heart movement and breathing movement. FIG. 2 is a visualization of a typical reconstruction of a coronary sinus (shown highlighted) with a reconstruction method of the type mentioned at the start having been applied.

Different approaches to the reconstruction of dynamically moved objects such as a coronary vessel system for example are possible with the aid of the filtered back projection.

One method is what is referred to as “gating” of the projection data. In this case an identification of the movement state of the object to be reconstructed is known (movement phase). Then, depending on the gating strategy, only the projections which are most similar to a specific reference movement phase are selected for the reconstruction. Basically two approaches exist for this. The first option of a reconstruction with a subset of the projection data (projection gaps) leads to a reduced image quality. The second option is the recording of further projections until all gaps are almost filled. A further method is movement correction. Mostly the change in the object over time is not known. This will be estimated from the projection data (estimation of the movement). With filtered back projection a target movement phase is then reconstructed in which the movement difference is compensated for for each projection image.

The methods described at the outset are disadvantageous in that a number of consecutive processing steps must be applied in a time-intensive way.

### SUMMARY OF THE INVENTION

The underlying object of the invention is to specify an improved method for three-dimensional image reconstruction as well as an associated imaging device by comparison with these previous methods.

This object is achieved by the features specified in the independent claims. Advantageous developments of the invention are specified in the dependent claims.

The subject matter of the invention is a method for three-dimensional image reconstruction of a dynamically moved object from projection data of an imaging device as well as an associated imaging device, especially a conventional x-ray device, a C-arm x-ray device or a computer tomography device. The inventive method comprises the following steps:
a) Determination of a reference volume from the projection data which simulates a similar static object instead of the dynamically moved object,
b) Assignment of the projection data to at least two disjoint consistent subsets, with a volume part reconstruction of the dynamically moved object being undertaken for each subset from projection data assigned to the subsets,
c) Application of a transformation to the respective volume part reconstructions, with the transformation representing a dynamic movement of the object,
d) Comparison of the volumes of the transformed part reconstructions with a corresponding part of the reference volume in each case, whereby, depending on the comparison result, step c) may be at least partly repeated, and
e) Summing of the volume part reconstructions resulting from step c) and d) to form a complete three-dimensional image reconstruction.

Depending on the above-mentioned comparison result, step c) can be at least partly repeated with a corrective transformation.

A further aspect of the invention is an imaging device, embodied with modules for three-dimensional image reconstruction from projection data in accordance with the above-mentioned method.

In an advantageous manner the invention describes a method for reconstruction of dynamically moved objects or objects able to change over time from projection data which is integrated into a method for filtered back projection if necessary. This enables the extra time spent in undertaking the reconstruction to be reduced.

A further advantage obtained from the invention is that movement artifacts are reduced in the reconstruction of dynamically moved objects.

Advantageously the object can represent a coronary vessel system which is contrasted if necessary.

Expediently the projection data can be filtered beforehand.

A further advantageous development of the invention makes provision for the comparison in step d) to be undertaken with the aid of maximum values from a quality function which is applied to the volumes compared in step d).

For determining the reference volume a tomographic and/or symbolic reconstruction from at least a part of the projection data can be applied.

The disjoint consistent subsets from step b) can be represented by single-element subsets.

Advantageously a transformation can be applied in step c) with the aid of checkpoints distributed over the object.

### BRIEF DESCRIPTION OF THE DRAWINGS

An exemplary embodiment of the invention is described in greater detail with reference to a drawing.

The drawing shows the following figures:

FIG. 1 a diagram for the execution sequence of the inventive method,

FIG. 2 a visualization of a reconstruction of a coronary sinus by means of filtered back projection in the way described at the outset and

FIG. 3 a visualization of a reconstruction of the coronary sinus in accordance with the invention.

### DETAILED DESCRIPTION OF THE INVENTION

As a concrete application the invention is represented by an example of the reconstruction of a contrasted part of a heart vessel system—the coronary sinus and the adjacent branch-off vessels.

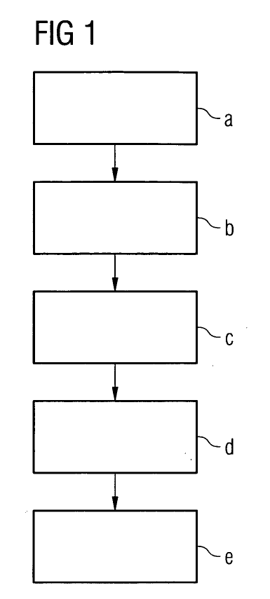

FIG. 1 shows a diagram for the execution sequence of the inventive method with the steps of the inventive method described below being identified by the letters a) to e):

a) A reference volume R, which contains information about the object to be reconstructed and the movement phase to be reconstructed, is created from the projection data P. This can be done in diverse ways and will be chosen individually. Examples are the tomographic or symbolic reconstruction from all projection data or from a subset. Also conceivable are postprocessing steps such as windowing, threshold value formation, segmentation or similar. If the object movement is divided up into phases, this information can then be included in the selection of the projection data.

Movement phases in the form of relative heart phases which can be created from an EKG signal are available for the reconstruction of the coronary sinus. The reference volume is reconstructed from a subset of the projection data in the heart rest phase with the FBP. As a rule projections from the area between 60-85% are selected. These can be represented with the visualization software, if necessary windowed and cut to shape.

Using an iterative application of the algorithm the reference volume can be improved. This means that the reference volume of the i+1th iteration is created from the result of the ith reconstruction. This can be applied in the example of the coronary sinus.

b) The projection data is divided into disjoint and consistent subsets P={P₁, . . . , Pₙ}. Let F(Pᵢ) be the part reconstruction of the projection data in Pᵢ. This can in its turn be determined dynamically or statically. The aim of this step is to find consistent subsets which are as large as possible.

For the coronary sinus reconstruction there is a breathing and a heart movement. In order to obtain consistent subsets in relation to the heart phases, the heart phases will be divided up into K equal-size intervals and the projections assigned in accordance with their heart phase. The part reconstructions are obtained by a recursive call of the algorithm specifically for correction of the breathing movement. In such cases the projections are divided up into single-element subsets. The transformation (see next step) is a strict translation which is restricted to one movement within the image plane. The target function and the reference volume remain unchanged. The part reconstruction obtained is thus breath-corrected and restricted to one heart phase area.

c) The definition of a transformation T(Θ), which is able to describe the movement of the object to be reconstructed using parameters Θ. Applied to a part reconstruction F(Pᵢ), this is modified in accordance with the parameterization. Examples are a strict movement in the coordinate directions, a rotation, a scaling or a free deformation. When selecting T it should be ensured that where possible this describes a movement which is unique. This mostly depends on the size of the subsets Pᵢ. If for example |Pᵢ|=1, no downwards movement along the direction of projection can be determined, which is why this degree of freedom should remain unconsidered where possible.

The heart vessel movement is characterized by a global up and down movement of the vessel tree over the heart cycle. This is described by a translation in the coordinate system. To take account of local deformations of the vessel tree, checkpoints distributed evenly over the volume are used, to which displacement vectors are assigned. For positions between the checkpoints displacement vectors from the surrounding neighbors are interpolated. This is done for example using a linear or B-spline interpolation.

As already explained above, only single-element subsets are used for the breathing movement. The breathing movement is assumed to be strict. Only a translation within the image plane and not in the camera direction (downwards) is possible.

d) The definition of a quality function λ(R,T(Θ){F(Pᵢ)}), which describes the match between the reference volume R and the transformed filtered back projection T(Θ){F(Pᵢ)}. An optimally transformed part reconstruction of the search area of the parameter set Θ maximizes λ. Let this optimally transformed part reconstruction be T(\hat{Θ}){F(Pᵢ)}. For the coronary sinus it is useful for the intensity of the back projections to be maximized at bright points of the reference volume R. This is achieved by the following target function:

$${λ}_{CS} ( R , T ( Θ ) \{ F ( {P}_{i} ) \} ) = {\sum }_{j} R ( j ) \cdot T ( Θ ) \{ F ( {P}_{i} ) \} ( j ) .$$

e) A complete reconstruction is provided by summing the transformed part reconstructions:

$$U = {\sum }_{i} T ( \hat{Θ} ) \{ F ( {P}_{i} ) \} .$$

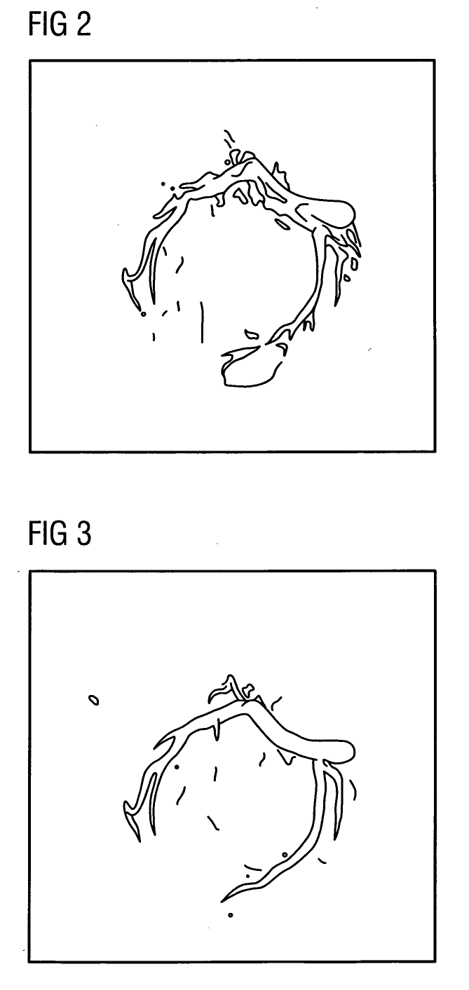

FIG. 3 shows a process visualization of a reconstruction of the coronary sinus (highlighted) in accordance with of the above-described method. In this figure it can be seen that the artifacts and unsharp areas shown in FIG. 2 have been compensated for or have disappeared in FIG. 3.

1.-8. (canceled)

9. A method for a three-dimensional image reconstruction of a dynamically moved object from projection data of an imaging device, comprising:
determining a reference volume from the projection data simulating a similar static object instead of the dynamically moved object;
assigning the projection data to a plurality of disjoint consistent subsets;
reconstructing volume parts of the dynamically moved object for the subsets from the projection data assigned to the subsets;
applying a transformation to the reconstructed volume parts, the transformation representing a dynamic movement of the object;
comparing the transformed reconstructed volume parts with corresponding volume parts of the reference volume for matching;
reapplying the transformation if necessary based on the comparison; and
summing the transformed reconstructed volume parts to form the three-dimensional image reconstruction.

10. The method as claimed in claim 9, wherein the dynamically moved object comprises a heart vessel system.

11. The method as claimed in claim 10, wherein the heart vessel system is contrasted

12. The method as claimed in claim 9, wherein the projection data is filtered beforehand.

13. The method as claimed in claim 9, wherein the comparison for the matching is determined by a maximum value of a quality function.

14. The method as claimed in claim 9, wherein the reference volume is determined by a tomographic or symbolic reconstruction from the projection data.

15. The method as claimed in claim 9, wherein the disjoint consistent subsets comprises single-element subsets.

16. The method as claimed in claim 9, wherein the transformation is applied by checkpoints distributed over the dynamically moved object.

17. An imaging device, comprising:
an imaging recording device that records projection data of a dynamically moved object; and
a computing device that:
determines a reference volume from the projection data simulating a similar static object instead of the dynamically moved object,
assigns the projection data to a plurality of disjoint consistent subsets,
reconstructs volume parts of the dynamically moved object for the subsets from the projection data assigned to the subsets,
applies a transformation to the reconstructed volume parts, the transformation representing a dynamic movement of the object,
compares the transformed reconstructed volume parts with corresponding volume parts of the reference volume for matching,
reapplies the transformation if necessary based on the comparison, and
sums the transformed reconstructed volume parts to form the three-dimensional image reconstruction.

18. The imaging device as claimed in claim 17, wherein the imaging device comprises an x-ray device, a C-arm x-ray device, or a computer tomography device.
